(19) United States
(12) Patent Application Publication
(10) Pub. No.: US 2023/0015225 A1
(43) Pub. Date: Jan. 19, 2023

(54) TOPICAL PHARMACEUTICAL COMPOSITIONS FOR TREATING ONYCHOMYCOSIS

(71) Applicant: GLAXOSMITHKLINE CONSUMER HEALTHCARE HOLDINGS (US) LLC, Wilmington, DE (US)

(72) Inventors: Pallav Arvind BULSARA, Warren, NJ (US); David John MOORE, Warren, NJ (US); Alireza SHALVIRI, Warren, NJ (US)

(21) Appl. No.: 17/787,187

(22) PCT Filed: Dec. 17, 2020
(86) PCT No.: PCT/US2020/065498

§ 371 Date: Jun. 17, 2022

Related U.S. Application Data
(60) Provisional application No. 62/951,070, filed on Dec. 20, 2019.

(51) Int. Cl. A61K 31/23; A61K 9/00; A61K 31/137; A61K 45/06; A61K 31/573; A61K 31/593; A61K 47/10; A61K 47/26; A61P 31/10

(52) U.S. Cl. CPC A61K 31/23; A61K 9/0014; A61K 31/137; A61K 31/573; A61K 31/593; A61K 45/06; A61K 47/10; A61K 47/26; A61P 31/10

(57) ABSTRACT

The present invention relates to topical pharmaceutical compositions for treatment or prophylaxis of onychomycosis, and other fungal conditions caused by Trichophyton rubrum and/or Trichophyton mentagrophytes, comprising an effective amount of an amine-based anti-fungal compound, such as terbinafine or a pharmaceutically acceptable salt thereof, and monocaprin, in certain proportions.

### FIELD OF THE INVENTION

The present invention relates to pharmaceutical compositions for topically treating onychomycosis.

### BACKGROUND OF THE INVENTION

Onychomycosis, also known as tinea unguium, is a fungal disease of the nail unit caused by dermatophytes, yeasts or non-dermatophye molds. Common organisms which infect the nail unit (which comprises the nail plate and tissues around and under it) include dermatophytes Trichophyton rubrum and Trichophyton mentagrophyte and the yeast,Candida albicans.

The signs and symptoms of the disease include split, thickened, hardened, and rough nail plates, and partial separation of the nail plate from the nail bed (i.e. the skin beneath the nail plate) creating an air gap in some areas.

The nail plate is thick, hard, and dense, and represents a formidable barrier to anti-fungal drug penetration. Although nail material is similar in various ways to the stratum corneum of the skin, the nail is composed primarily of hard keratin which is highly disulfide-linked and is approximately 100-fold thicker than stratum corneum. Widely used amine-based anti-fungal agents such as terbinafine are known to bind extensively to nail keratin, hindering drug penetration; in onychomycosis the thickening of the nail plate further hinders drug delivery.

Because of the difficulty in obtaining clinically effective concentrations of drug transungually (i.e. across the nail) to the nail bed, onychomycosis has typically been treated either with systemic medications, or with topical medications preceded by nail removal. However, systemic treatment is often not satisfactory due to liver toxicity or drug interactions, coupled with therapeutic regimens extending over weeks and even months. Topical anti-fungal treatment (e.g., with miconazole or ketoconazole) preceded by nail removal is also a measure most individuals would prefer to avoid if a less drastic topical therapeutic method were available. FDA-approved products for the topical treatment of onychomycosis include Penlac® Nail Lacquer (ciclopirox topical Solution, 8%) and JubliaⓇ (efinaconazole topical solution, 10%). However, none of these therapies has proven to be consistently effective.

U.S. Published Pat. Application No. 2011/305646 (Lenn et al.), which is incorporated by reference, discloses emulsion compositions for treating T. rubrum fungal infections comprising a fatty acid monoester of glycerol as the pharmaceutically active agent, optionally with a second pharmaceutically active agent.

There remains a significant need for a topical pharmaceutical composition that provides enhanced transungual penetration of an anti-fungal agent and thus improved therapeutic effectiveness.

### SUMMARY OF THE INVENTION

We have achieved unexpected improvements in transungual drug penetration by combining an amine-based anti-fungal agent with monocaprin, a 1-monoglyceride of capric acid, (a C₁₀saturated fatty acid), in certain proportions in a topically administrable vehicle. In zone of inhibition (ZOI) assays predictive of in vivo efficacy in the treatment of onychomycosis, this combination has yielded unforeseen improvements in achieving fungal eradication. For example, a composition of the invention combining 1% terbinafine and 1.4% monocaprin in a topically administrable vehicle demonstrates a zone of inhibition against T. rubrum that is statistically greater than the combined zones of terbinafine-only and monocaprin-only compositions, indicating the synergistic anti-fungal activity of the combination.

We have also discovered that monocaprin is superior to other, structurally similar monoglycerides, such as glyceryl monocaprylate, the monoglyceride of caprylic acid (C₈) (“monocaprylate”); glyceryl monolaurin, the monoglyceride of lauric acid C₁₂) (“monolaurin”); as well as undecylenic acid (unsaturated C11 fatty acid), in facilitating nail penetration and anti-fungal activity by an amine-based anti-fungal agent.

While not intending to be bound thereby, we postulate that the monocaprin, particularly when present in certain defined proportions relative to the amine-based anti-fungal agent, serves the function of reducing or eliminating amine/keratin interactions that hinder transungual penetration, either by competitively binding keratin in the nail and/or by forming micellar structures around the amine. We also theorize that monocaprin may be exerting a surfactant effect in the compositions of the invention, either by lowering interfacial tension, narrowing colloidal distribution, and/or reducing average colloidal size.

By “amine-based fungal agent” is meant a pharmaceutically or physiologically acceptable compound having anti-fungal activity which comprises an amine group, and the pharmaceutically acceptable salts thereof. Examples of amine anti-fungal agents include allylamines such as terbinafine, naftifine and as benzylamines such as butenafine and the pharmaceutically accetable salts thereof.

Thus the present invention provides topical compositions for the treatment of onychomycosis or other fungal conditions of the nail unit, in particular caused by T. rubrurm and/or T. mentagrophytes, comprising the combination of a therapeutically effective amount of an amine-based anti-fungal agent and monocaprin in a topically administrable vehicle.

The present invention also provides methods for the treatment of onychomycosis or other fungal conditions, in particular caused by T. rubrum and/or T. mentagrophytes, in a mammalian (e.g., human) patient in need thereof, the method comprising topically administering to the affected area (e.g., the nail plate and/or skin of the nail unit if indicated), a composition according to the invention.

According to a further aspect, the present invention relates to the use of the compositions described herein for the preparation of a medicament for the treatment of onychomycosis or other fungal conditions, in particular caused by T. rubrum and/or T.mentagrophytes, in a mammalian (e.g., human) patient in need thereof.

### BRIEF DESCRIPTION OF THE DRAWINGS

FIGS. 1, 2, 3, and 4 depict the results of full scale TurChubⓇ zone of inhibition (ZOI) assays wherein the vertical bars measure the mean distance of dermatophyte growth on T. rubrum-innoculated Sabouraud dextrose agar (SDA) growth medium in cells mounted with full thickness nails the surface of which was treated with a composition of the invention or a comparator (e.g., LamisilⓇ cream, LamisilⓇ Dermgel, LamisilⓇ Once, JubliaⓇ or PenlacⓇ). An infected control; non-infected control; and vehicle alone were also tested. The horizontal bar at the top of the chart represents the maximum range of length (ca. 3.2 - 4.0 cm) of the agar within the TurChubⓇ cell.

FIG. 5 depicts ATP recovered from T. rubrum-infected nails (wherein the bars represent the percentage of luminescence units compared to the infected control) following a single treatment with test formulation; vehicle alone; infected control; or non-infected control.

FIG. 6 depicts cumulative penetration (µg/cm²) of terbinafine across pre-sliced bovine hoofs mounted in Franz diffusion cells equipped with nail adaptors, from a composition of the invention comprising 1% terbinafine and 1.4% monocaprin; or a “control” composition comprising comprising 1% terbinafine in 100% ethanol, or comparators LamisilⓇ Gel and LamisilⓇ Cream, into a receptor medium, as determined by HPLC.

FIG. 7 depicts terbinafine affinity to bovine hoof-derived keratin from either a composition of the invention comprising a solution of 1% terbinafine and 1.4% monocaprin or a “control” composition comprising 1% terbinafine in 100% ethanol (each bar representing the mean ± SD of two replicates).

### DETAILED DESCRIPTION OF THE INVENTION

The compositions of the invention comprise the combination of an amine-based anti-fungal agent and monocaprin in a topically administrable vehicle.

Monocaprin is a saturated, C₁₀ fatty acid monoester of glycerol having the structural formula:

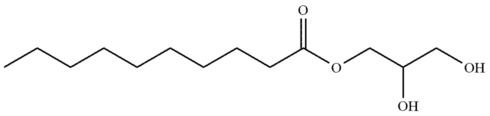 [SMILES: CCCCCCCCCC(=O)OCC(O)CO]

and is also known as 1-decanoyl-rac-glycerol, i.e. glycerol monocaprate, or alternatively, the 1-monoglyceride of capric acid, i.e. 1-monocaprin.

The amine-based anti-fungal agent is preferably an allylamine, most preferably terbinafine or its pharmaceutically acceptable salts. (Unless otherwise indicated, the term, “allylamine,” as used herein shall hereinafter refer collectively to the allylamine and its pharmaceutically acceptable salts.)

Typically, the concentration of the amine-based anti-fungal agent will be in a therapeutically effective amount, i.e. from 0.0001 to 30% or higher by weight of the composition. By way of illustration, the compound may be present in the composition in an amount from 0.1 to 10%, more preferably from 0.1 to 5%, e.g., 1 to 5%, even more preferably from 0.1 to 2.5%, even more preferably 0.1 to 1.5%, e.g., 1%, all percentages being with reference to the free base, and expressed as weight percentages based on the total composition.

The monocaprin is preferably present in an amount from 0.1% to 10% by weight, preferably 0.1% to 5%, more preferably 0.1% to 1.5%, based on the total composition.

Unexpected synergy in achieving transungual penetration and anti-fungal activity of the amine-based anti-fungal agent has been discoverered when the amine (in particular, terbinafine) and monocaprin are present over a range, based on weight percent in the total composition, of most broadly, 5:1to 1:6 (amine free base : monocaprin). Preferably the range of ratios is between 3.5:1 to 1:1.4 (amine free base: monocaprin.) Examples of suitable wt% ratios may be about 1:1, 1:1.4, and 1:1.5 (amine free base: monocaprin), based on the total composition. Another suitable range, based on weight percent in the total composition, is 1:1.4 to 1:5.7 (amine free base: monocaprin).

Furthermore, the results of our ZOI studies with compositions comprising terbinafine and monocaprin indicate that by maintaining the two in the proportions recited above, anti-fungal activity can be achieved with relatively low levels of terbinafine, beneficially minimizing patient exposure to the anti-fungal drug and its potential side effects. Thus, in compositions of the invention comprising an allylamine such as terbinafine or its pharmaceutically acceptable salt as the anti-fungal agent, the terbinafine is present in an amount which is at least 1 wt%, and more preferably, at least 1.4 wt.%, but preferably not exceeding 5 wt.%, i.e. 1-5 or 1.4.-5 wt.% (calculated as the free base in the total composition). Specific examples of suitable concentrations may be 1 wt.%, 1.375 wt.%, 2.5 wt.% and 5 wt.% based on the total composition.

### Topically Administrable Vehicle

The topically administrable vehicle of the composition of the invention is a pharmaceutically acceptable vehicle in which the amine-based anti-fungal agent and monocaprin, as well as any other ingredients of the formulation can be dissolved, suspended, disbursed, or emulsified.

### Volatilesolvent

It has beenfound suitable to employ a topically administrable vehicle comprising predominntly a volatile solvent; preferably, a volatile solvent which is water miscible.

As used herein, the term “volatile” means that the solvent evaporates from the surface of the nail when applied. Volatile solvents are compounds which have a measurable vapor pressure, which is preferably greater than 100 Pa at room temperature.

When the composition is administered to the nail and optionally the surrounding skin, evaporation of the volatile solvent leaves a “secondary composition” comprising a matrix of the allylamine anti-fungal agent, the monocaprin, and other ingredients of the composition.

It is preferred that the allylamine anti-fungal agent and monocaprin are soluble or miscible in the volatile solvent and can be dissolved, suspended, dispersed, or emulsified in the secondary composition that remains after the volatile solvent has evaporated or penetrated the nail. It is also suitable that any other ingredients of the composition also be dissolvable, suspendable, dispersible, or emulsifiable, in the secondary composition.

The secondary composition in a preferred embodiment remains in a liquid or semi-solid state after application to the nail and does not form a hard lacquer, shell, or film on the nail following application, which occurs by a process of solvent casting following evaporation of the volatile solvent, leaving behind a solid residue that forms the lacquer, shell or film which is lasting, hard, and/or adherent. Thus it is preferred that the composition is substantially free of polymeric film forming compounds, or if including such compounds that they be present in an amount below that which will result in the formation of a film or lacquer following application of the composition to the surface of a nail.

In a preferred aspect, the volatile solvent comprises a lower alcohol, more preferably a monohydric lower alcohol. Still more preferably, the lower alcohol is selected from a C₁₋₆ alkanol, such as methanol, ethanol, propanol, isopropanol, butanol, isobutanol, sec-butanol, pentanol, and the like, as well as mixtures thereof.

In addition to monohydric lower alcohols, the solvent may also be selected from butoxy ethanol, acetone, ethyl acetate, butyl acetate, and mixtures thereof.

In a preferred embodiment, the volatile solvent is ethanol, especially ethanol 100% v/v.

In another aspect, the lower alcohol comprises a diol or triol. Suitable diols and triols include, but are not limited to, glycerol, ethylene glycol, propylene glycol, butanediol, butynediol, pentanediol, hexanediol, octanediol, neopentyl glycol, 2 -methyl-1,3 -propanediol, diethylene glycol, triethylene glycol, tetraethylene glycol, dipropylene glycol, dibutylene glycol, propylene glycol, butanetriol, pentanetriol, hexanetriol, and the like, and mixtures thereof.

In one embodiment, the diol is selected from ethylene glycol,propyle glycol, and mixtures thereof.

According to an embodiment, the volatile solvent is present in the compositions of the invention in an amount from 50% to 99.85% by weight, e.g., for example, from 60% to 90% by weight, e.g., 70-90 wt.%, or 75-90 wt.% based on the composition.

### Water

The compositions of the invention also generally comprise at least a small amount of water. Generally, the amount of water in the composition ranges from 1-10 wt. %, preferably 1-5 wt.%.

### Co-Solvent

The topically administrable vehicle may also comprise a co-solvent which is miscible with the aqueous-alcoholic phase.

Co-solvents suitable for this purpose are, for example, polyhydric alcohols, such as glycerine, ethylene glycol or propylene glycol, especially poly-lower alkylene glycol (e.g., polyethylene glycol or polypropylene glycol) having a chain length of from approximately 200 to approximately 6000, preferably from approximately 300 to approximately 1500, units.

In a preferred aspect, the compositions comprise, as the volatile solvent, a lower alcohol, more preferably a monohydric lower alcohol, such as ethanol, and as co-solvent, a polyhydric alcohol, especially propylene glycol.

The co-solvent when present is preferably present in an amount from 5 to 10% by weight of the composition.

### Additional Excipients

The compositions of the invention may comprise additional excipients known to the art, including, e.g., surfactants, humectants, emollients, pH adjusting agents, preservatives, antioxidants, lubricants, penetration enhancers, fragrance, colorants, gelling agents, radical scavengers, etc., and combinations and mixtures thereof.

### Surfactants

Any surfactant or group of surfactants that is suitable for dermatologic applications is suitable for use in the compositions of the invention. Such surfactants may function as wetting agents, emulsifiers or solubilizers. The surfactants may be nonionic, anionic, cationic, zwitterionic, amphoteric, or ampholytic.

Examples of non-ionic surfactants include polyoxyethylene sorbitan fatty acid esters such as polysorbate 20, 40, 60 and 80; sorbitan esters such as sorbitan monolaurate, sorbitan monooleate, sorbitan monostearate, and sorbitan trioleate; polyoxyethylene alkyl ethers such as Brij 30, Brij 97, Emulgen 104P, 210P, 200 and Ethylan 253, 254, 256, and 257; and polyoxyethylene castor oil derivatives such as polyoxyl 35 castor oil.

Traditional anionic surfactants include ammonium lauryl sulfate, sodium lauryl sulfate (sodium dodecyl sulfate, SLS, or SDS), and the related alkyl-ether sulfates sodium laureth sulfate (sodium lauryl ether sulfate or SLES), docusate (dioctyl sodium sulfosuccinate), perfluorooctanesulfonate (PFOS), alkyl-aryl ether phosphates and alkyl ether phosphates.

Traditional cationic surfactants include cetrimonium bromide (CTAB), cetylpyridinium chloride (CPC), and benzalkonium chloride (BAC).

Zwitterionic surfactants include cocamidopropyl hydroxysultaine and betaines such as cocamidopropyl betaine.

Suitably, the surfactant is present in the composition in an amount from 0.01% to 10% by weight. Preferably the surfactant is present in an amount from 0.25% to 2% by weight.

### Humectants

Non-limiting examples of humectants useful in the compositions of the invention include glycerol, sorbitol, maltitol, polydextrose, triacetin, propylene glycol, polyethylene glycol (PEG) esters including PEG-20 stearate, PEG-40 stearate, PEG-150 stearate, PEG-150 distearate and PEG-100 stearate, alkoxylated alcohols including laureth-12, ceteareth-20, laureth-23, glycereth-7, glycereth-12, glycereth-26, PEG-4, PEG-6, PEG-8, PEG-12, PEG-32, PEG-75, PEG-150, and combinations and mixtures thereof.

In general, the compositions may comprise 0.1% to 10%, e.g., from 0.5% to 5%, by weight of a humectant.

### Emollients

In some embodiments, the compositions may include one or more emollients. In certain embodiments, the one or more emollients may be fatty esters, fatty alcohols, or combinations thereof including, but not limited to, diisopropyl adipate, oleyl alcohol, lanolin, isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, polyoxypropylene (5) poloxyethylene (20) cetyl ether (PPG-5-Ceteth-20), 2-ethylhexyl isononoate, 2-ethylhexyl stearate, C₁₂ to C₁₆ fatty alcohol, C₁₂ to C₁₆ fatty alcohol lactate, isopropyl lanolate, 2-ethyl-hexyl salicylate, and combinations and mixtures thereof.

The one or more emollients may include 1-hexadecanol, acetylated lanolin, behenocyl dimethicone, C₁₂ to C₁₅ alkyl benzoate, cetearyl octanoate, cocoglycerides, dicaprylate/dicaprate dimethicone copolyol, dimethiconol, dioctyl adipate, glyceryl stearate, isocetyl alcohol, isohexadecane, isopentylcyclohexanone, isopropyl palmitate, lauryl lactate, mineral oil, methoxy peg-22/dodecyl glycol copolymer, myristyl lactate, ocryldodecyl neopentanoate, octyl cocoate, octyl palmitate, octyl stearate, octyldodecyl neopentanoate, polyglyceryl-4 isosterate, polyoxyl 40 stearate, polyoxymethylene urea, potassium sorbate, propylene glycol, propylene glycol isoceth-3 acetate, and propylene glycol myristyl ether acetate.

A preferred emollient is CERAPHYL 41, which comprises a mixture of C₁₂-C₁₅ alcohol lactates, available from ISP Van Dyk Inc. Another preferred emollient is a fatty alcohol such as cetyl alcohol.

The emollient may be provided in any suitable amount, for example, from 0.01% to 10% by weight, preferably from 0.25% to 2% by weight.

### PH Adjusting Agents

Suitable pH adjusting bases include amines, bicarbonates, carbonates, and hydroxides such as alkali or alkaline earth metal hydroxides (e.g., sodium hydroxide), as well as transition metal hydroxides. In another embodiment, the pH adjusting agent is an acid, an acid salt, or mixtures thereof. According to yet another embodiment, the pH adjusting agent is a buffer. Suitably, the buffer is selected from citrate/citric acid, acetate/acetic acid, phosphate/ phosphoric acid, formate/formic acid, propionate/propionic acid, lactate/lactic acid, carbonate/carbonic acid, ammonium/ammonia, edetate/edetic acid, and combinations and mixtures thereof.

According to an embodiment, the pH adjusting agent is present in an amount from 0.01% to 10% by weight. According to another embodiment, the pH adjusting agent is present in an amount sufficient to adjust the pH of the composition to between 4 and 6.5.

### Preservatives

The present pharmaceutical compositions may further comprise a preservative, non-limiting examples of which may include benzyl alcohol, diazolidinyl urea, methyl paraben, ethyl paraben, propyl paraben, butyl paraben, phenoxyethanol, sorbic acid, benzoic acid, salts thereof, and combinations and mixtures thereof.

In an embodiment, the present compositions comprise a preservative in an amount from 0.01% to 2% by weight.

### Additional Active Agents

According to an embodiment of the present invention, the pharmaceutical compositions may comprise a second pharmaceutically active, topically administrable agent.

In an embodiment, the second pharmaceutically active agent is selected from the group consisting of an antibacterial agent, corticosteroid and vitamin D analogue.

Suitably, the antibacterial agent is selected from the group consisting of gentamicin, neomycin, streptomycin, cefpodoxime proxetil, clindamycin, lincomycin, erythromycin, bacitracin, gramicidin, vancomycin, doxycycline, minocycline, oxytetracycline, tetracycline, fosfomycin, fusidic acid, mupirocin, sulfacetamide, metronidazole, dapsone, triclosan, quaternary ammonium salts, silver sulfadiazine, and salts and esters thereof.

Although the compositions of the invention may be used to treat various diseases and disorders of the skin or mucous membranes, they are most advantageously used to treat conditions involving the nails of the hands or feet. The compositions and methods of the invention are found to provide increased penetration of the amine-based antifungal ingredient into and through the nail and to the nail bed. The compositions of the invention may be used effectively to treat diseases and disorders in humans or in other animals, such as cats, dogs, horses, cattle, sheep, goats, pigs, and birds. In human and in veterinary patients, the compositions of the invention may be used, depending on the particular animal treated, to treat conditions involving nails, hooves, horns, or beaks.

The compositions of the invention are especially well suited for the treatment of onychomycosis and other disorders of the nail and nail bed. The compositions are particularly effective in treating the specific dermatophyte Trichophyton rubrum. According to an embodiment, the present invention relates to a method of treating a fungal disorder caused by a T. rubrum infection. In one embodiment, the fungal disorder is selected from the group consisting of onychomycosis, tinea pedis (athlete’s foot), tinea cruris (groin) and dermatophytosis (ringworm). According to one embodiment, the fungal condition is onychomycosis. The present compositions are also effective in treating fungal conditions caused by Trichophyton mentagrophytes and Epidermophyton floccosum.

In use the composition is administered to the surface of the nail and surrounding tissue by any means by which the composition may be applied. The method of application may vary depending on the physical state of the composition, whether it is in a liquid, semisolid, or solid form, and on the viscosity of the composition if it is a liquid. Thus, for example, the composition may be rubbed, painted, dabbed, dripped, sprayed, wiped, spread, or poured onto the affected nail and surrounding tissues, or utilized as a soak.

Frequency of treatment and duration of therapy will vary depending on several factors, including the condition that is being treated, the identity and concentration of the active ingredient in the composition, and constituents of the composition other than the active ingredient.

Typically, the frequency of treatment will be twice daily to once weekly, and preferably once daily.

The preferred duration of topical treatment is typically at least about 36 weeks and preferably longer, such as, e.g., 40 weeks or 48 weeks. The preferred criterion for treatment efficacy is complete cure (i.e. fungal eradication), which can be assessed at the end of treatment, but is preferably assessed at 4 to 12 weeks after the end of treatment, most preferably at 4 weeks after the end of treatment.

Combination Therapy. In another embodiment of the invention, the present compositions may be used in combination with an additional (separate) dosage form to enhance the treatment of the fungal condition. This additional dosage form may be applied or taken at the same time as the present compositions i.e. concomitantly. Alternatively, one of the present compositions and the additional dosage form is administered in the morning and the other is administered in the evening (or vice versa).

Thus, for example, the present composition is administered as a combination with a separate oral composition comprising an anti-fungal agent. Exemplary anti-fungal agents suitable for use in the oral composition include, but are not limited to, terbinafine, albaconazole, pramiconazole, itraconazole, griseofulvin or fluconazole.

According to an alternative embodiment, the present pharmaceutical compositions are used as a maintenance therapy. Maintenance therapy is initiated following substantial or complete alleviation of the symptoms of the fungal condition following primary treatment.

### Definitions

As used herein, the terms “administered”, “administering” and “administration,” refer to any method which, in sound medical practice, topically delivers the composition to a patient in such a manner as to provide a therapeutic effect.

The terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

Throughout the application, descriptions of various embodiments use “comprising” language, however it will be understood by one of skill in the art, that in some specific instances, an embodiment can alternatively be described using the language “consisting essentially of” or “consisting of”.

As used herein, the phrases an “effective amount” or a “therapeutically effective amount” of an active agent or ingredient, or pharmaceutically active agent or ingredient, which are synonymous herein, refer to an amount of the active agent sufficient to have a therapeutic effect upon administration. An “effective amount” or “therapeutically effective amount” of the amine-based anti-fungal agent is expected to treat or prevent, and preferably to eradicate, the fungal condition, e.g., onychomycosis. Effective amounts will vary with the particular condition being treated, the severity of the condition, the duration of the treatment, and the specific components of the composition being used.

As used herein, the term “pharmaceutically acceptable salts” refers to salts that are pharmaceutically acceptable and that possess the desired pharmacological activity of the parent compound. Such salts include: (1) acid addition salts, formed with acids such as, for example, acetic acid, benzoic acid, citric acid, gluconic acid, glutamic acid, glutaric acid, glycolic acid, hydrochloric acid, lactic acid, maleic acid, malic acid, malonic acid, mandelic acid, phosphoric acid, propionic acid, sorbic acid, succinic acid, sulfuric acid, tartaric acid, naturally and synthetically derived amino acids, and mixtures thereof; or (2) salts formed when an acidic proton present in the parent compound is either (i) replaced by a metal ion e.g. an alkali metal ion, an alkaline earth metal ion, or an aluminium ion; or (ii) protonates an organic base such as, for example, ethanolamine, diethanolamine, triethanolamine, tromethamine and N-methylglucamine.

As used herein, the term “substantially free” of a specified component refers to a composition with less than 1% of the specified component.

As used herein, a “treatment” or “treating” of a condition encompasses alleviation of at least one symptom thereof, a reduction in the severity thereof, or the delay, prevention or inhibition of the progression thereof. Treatment need not mean that the condition is totally cured. A useful composition herein need only to reduce the severity of a condition, reduce the severity of symptoms associated therewith, provide improvement to a patient’s quality of life, or delay, prevent or inhibit the onset of a condition.

For the purposes of better understanding the present teachings and in no way limiting the scope thereof, unless otherwise indicated, all numbers expressing quantities, percentages or proportions, and other numerical values used in the specification and claims, are to be understood as being modified in all instances by the term “about”.

Other terms as used herein are meant to be defined by their well-known meanings in the art. Unless defined otherwise, all technical and scientific terms used herein have the same meaning as commonly understood to one of ordinary skill in the art to which the presently described invention pertains.

The following Examples are illustrative of the present invention and are not intended to be limitative thereof.

### Experimental Methods

In vitro TurChub® zone of Inhibition (ZOI) and ChubTur® infected human cadaver nail models.

The TurChub® ZOI model developed by Medpharm Ltd. was used for screening of formulations for transungual penetration and anti-fungal efficacy of terbinafine from compositions of the invention. The apparatus consists of a modified Franz-type cell whose: housing is designed to accommodate the natural curvature of a human nail while maintaining a suitable seal to divide the dosing chamber from the receiving chamber. The ChubTur® nail infected model (MedPharm Ltd.) was also used for testing of formulations on T. rubrum infected human cadaver nail samples. In this model ATP activity is used as a measure of fungus viability (i.e. higher ATP activity corresponding to more fungal viability and hence lower anti-fungal efficacy). The TurChub“ and ChubTur® models are described in R. Turner et al., ”A Novel Vehicle for Enhanced Drug Delivery Across the Human Nail for the Treatment of Onychomycosis,“ Int J Pharm Compd. 2016;20(1):71-80; L. Christensen et al., ”Evaluation of the ability of a novel miconazole formulation to penetrate nail by using three in vitro nail models,“ Antimicrob Agents Chemother. 2017;61(7):02554-16; and M. Brown et al. (2018) ”Use of in vitro performance models in the assessment of drug delivery across the human nail for nail disorders," Expert Opinion on Drug Delivery, 15:10, 983-989.

For the TurChub® assay, at t=0, a single dose of 10 µL of each test formulation was pipetted onto the dorsal side of full thickness individual nails and the nails were air dried for 10 minutes in a laminar flow cabinet. The nails were mounted in the TurChub® cell (n=6, mean ± SEM) and incubated for 7 days at 20-2.5° C. The efficacy of the test formulations was determined by measuring the length of the zone of inhibition of dermatophyte growth on T, rubrum-innoculated agar underside the nail. “infected” controls were inoculated with organism but not treated with test formulation. “Non-infected” controls were neither inoculated with T. rubrum nor treated with test formulation,

### ATP Assay

The ATP assay was used to generate calibration curves (to show linearity between ATP recovery and luminescence units over a typical experimental range) and to demonstrate that there was no significant quenching effect from the test formulations on the assay.

### In Vitro Bovine Hoof Permeation Study

Terbinafine permeation from formulations across pre-sliced bovine hoof (thickness 0.08 mm- 0.10 mm) was evaluated using a Manual Franz Diffusion IVRT Apparatus equipped with nail adaptors. A single 300 µl dose of each test formulation was applied to a hoof sample, and after 7 days cumulative drug permeation into a receptor medium of citrate buffer pH 5.0 with 0.01% sodium azide, was quantified using HPLC with a lower limit of quantifcation of 0.1 µg/ml.

### Terbinafine/Keratin Binding Assay

The affinity of terbinafine for bovine hoof-derived keratin when the terbinafine is in a composition of the invention also comprising monocaprin, as compared with a control (terbinafine in 100% ethanol) was determined by adding 3 ml of formulation to approximately 300 mg of the keratin in centrifuge tubes; placing the samples on oscillating plates for 9 days; and centrifuging the samples at 2,000 rpm for 10 minutes. Unbound drug in the supernatant was assayed to calculate the % terbinafine binding.

### Example 1

The formulations shown on Table 1 were tested in the Turchub® ZOI assay as described above.

Table 1

| | Test formulation | Anti-fungal agent | Formulation type |
|---|---|---|---|
| (1) | Terbinafine HCl and monocaprin | Terbinafine¹, 1% and monocaprin, 5.7% | Solution² |
| (2) | Lamisil® Cream (comparator) | Terbinafine, 1% | Cream |
| (3) | Lamisil® Dermgel (comparator) | Terbinafine, 1% | Gel |
| (4) | Lamisil® Once (comparator) | Terbinafine, 1% | Solution (film forming) |
| (5) | Jublia® Topical Solution (comparator) | Efinaconazole, 10% | Solution³ |
| (6) | Penlac® Nail Lacquer Topical Solution (comparator) | Ciclopirox (8%) | Lacquer⁴ |

¹ Terbinafine strength is with reference to the free base (indicated in brackets in Tables 2-4). ² The actives in the solution chassis described in Example 2. ³ Jublia® also comprises as inactive ingredients: alcohol, anhydrous citric acid, butylated hydroxytoluene, C₁₂₋₁₅ alkyl lactate, cyclomethicone, diisopropyl adipate, disodium edetate, and purified water. ⁴ Penlac® has a solution base comprising: ethyl acetate, isopropyl alcohol, and butyl monoester of poly[methylvinyl ether/maleic acid, in isopropyl alcohol.

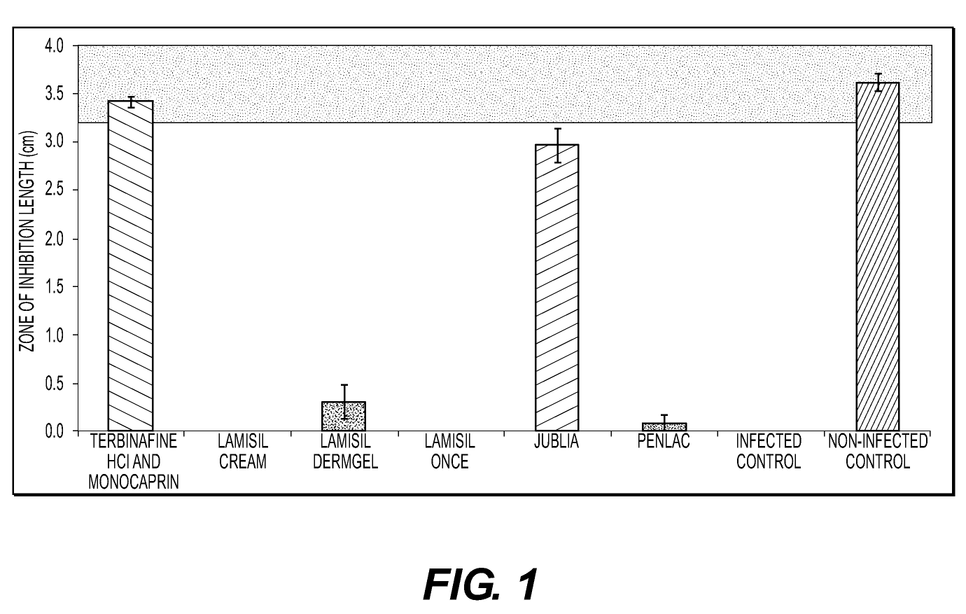

As shown in FIG. 1, test formation (1) of the invention, comprising terbinafine, 1% and monocaprin, 5.7%, resulted in a ZOI of 3.4 ± 0.1 cm, equivalent to total kill of T. rubrum in the TurChub® cell. There was no detectible ZOI following application of comparator formulations (2) Lamisil® Cream and (4) Lamisil® Once; however, there was a mean ZOI of 0.3 ± 0.2 cm for comparator formulation (3) i.e. Lamisil® Dermgel. The mean ZOI for comparators (5) Jublia® and (6) Penlac® were 3.0 ± 0.2 and 0.1 ± 0.1 cm, respectively.

When statistically compared as a whole population at the 95% confidence level, the mean ZOI for test formulation (1) of the invention and comparator (5) (Jublia®) were not statistically different (p ≥ 0.05); however, both were statistically different (p < 0.05) from the mean ZOI of the remaining test formulations.

The mean ZOI of the comparators other than Jublia® were not statistically different (p ≥ 0.05) from each other. The small ZOI, or no zone, for these formulations indicated relatively low anti-fungal efficacy through the nail.

The results demonstrate that the terbinafine/monocaprin composition of the invention and Jublia® outperformed (p < 0.05) the other comparator formulations with respect to anti-fungal efficacy.

Further, when compared as independent samples at the 90% confidence level, the terbinafine/ monocaprin composition of the invention outperformed Jublia® in anti-fungal efficacy (p < 0.05).

### Example 2

The Torchub® ZOI assay was used for testing of various formulations listed on Table 2, comprising as active ingredients, either terbinafine, 1% alone [(A) and (B)], monocaprin, 1.4% or 5.7%, alone [(C) and (D)), or the combination of terbinafine, 1% and monocaprin, 1.4% or 5.7%, [(E) and (F)], in a solution chassis.

Table 2

| Ingredients INCI (%w/w) | Test Formulation | | | | | |
|---|---|---|---|---|---|---|
| | (A) | (B) | (C) | (D) | (E) | (F) |
| terbinafine HCI¹ | 1.125 [1] | 2.812 [2.5] | | | 1.125 [1] | 1.125 [1] |
| monocaprin | | | 1.4 | 5.7 | 1.4 | 5.7 |
| solution chassis: | | | | | | |
| ethyl alcohol, 100% v/v | 81.325 | 79.638 | 81.05 | 76.75 | 79.92 | 75.625 |
| butylhydroxytoluene (BHT) | 0.05 | 0.05 | 0.05 | 0.05 | 0.05 | 0.05 |
| stearyl alcohol | 0.5 | 0.5 | 0.5 | 0.5 | 0 | 0.5 |
| cetyl alcohol | 1.1 | 1.1 | 1.1 | 1.1 | 1.6 | 1.1 |
| polysorbate 60 | 0.4 | 0.4 | 0.4 | 0.4 | 0.4 | 0.4 |
| propylene glycol | 10 | 10 | 10 | 10 | 10 | 10 |
| sodium Hydroxide 30% (w/w) | 0.5 | 0.5 | 0.5 | 0.5 | 0.5 | 0.5 |
| Water | 5 | 5 | 5 | 5 | 5 | 5 |

¹1.125 g Terbinafine HCl = 1 g Base Terbinafine

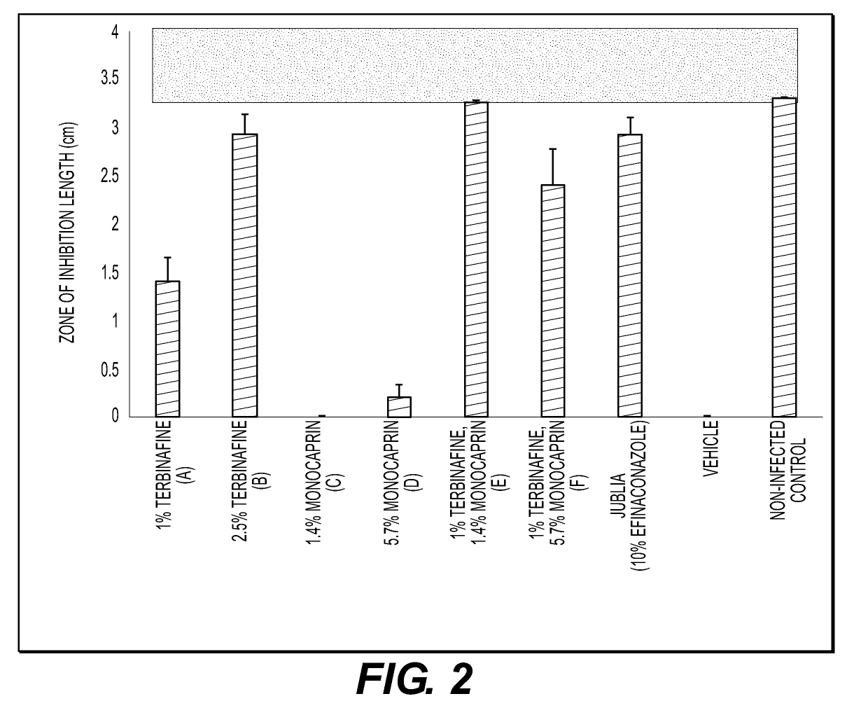

FIG. 2 shows the results of the ZOI investigations, using Jublia® as comparator. Monocaprin alone formulations, (C) and (D), exhibited some efficacy only at high monocaprin concentration (D). Terbinafine-only solution formulations, (A) and (B), showed improved efficacy compared to the Lamisil® comparators tested previously, with increased terbinafine concentration correlating with increased ZOI.

Notably, the ZOI for test formulation (E), comprising terbinafine, 1% and monocaprin, 1.4%, was statistically greater than the combined zones for test formulations (A) (terbinafine, 1%), and (E) monocaprin (1.4%)) (p < 0.05), indicating a synergistic anti-fungal effect of terbinafine combined with monocaprin. Furthermore, formulation (E) outperformed formulation (F), indicating that the ratio of terbinafine to monocaprin is an important factor as well.

### Example 3

The Torchub® ZOI assay was used to evaluate anti-fungal efficacy of terbinafine, 1% combined with either of monolaurin (C₁₂) [test formulation (H)], monocaprylate (C₈) [test formulation (K)], or undecylenic acid [test formulation (M)], in the solution chassis, as well as the solution chassis itself (“vehicle”), and compared with the ZOI results previously obtained for formulations (A), (C) and (E) of Example 2 and comparator Jublia®. Also tested were formulations comprising monolaurin [test formulation (G)] monocaprylate [test formulation (I)), and undecylenic acid [test formulation (L)), in the absence of terbinafine. The formulation details are provided in Table 3.

Table 3

| Ingredients INCI (%w/w) | Test Formulation | | | | | |
|---|---|---|---|---|---|---|
| | (G) | (H) | (I) | (K) | (L) | (M) |
| terbinafine HCl | - | 1.13 [1] | | 1.13 [1] | | 1.13 [1] |
| monocaprin | - | - | - | - | - | - |
| monolaurin | 1.4 | 1.4 | - | - | - | - |
| monocaprylate | - | - | 1.4 | 1.4 | - | - |
| undecylenic acid | - | - | - | - | 10 | 10 |
| ethyl alcohol, 100% v/v | 81.05 | 79.92 | 81.05 | 79.92 | 72.95 | 71.82 |
| butylhydroxyanisole (BHA) | 0.05 | 0.05 | 0.05 | 0.05 | 0.05 | 0.05 |
| stearyl alcohol | 0.5 | 0.5 | 0.5 | 0.5 | 0.5 | 0.5 |
| cetyl alcohol | 1.1 | 1.1 | 1.1 | 1.1 | 1.1 | 1.1 |
| polysorbate 60 | 0.4 | 0.4 | 0.4 | 0.4 | 0.4 | 0.4 |
| propylene glycol | 10 | 10 | 10 | 10 | 10 | 10 |
| sodium hydroxide 30% (w/w) | 0.5 | 0.5 | 0.5 | 0.5 | 0.5 | 0.5 |
| Water | 5 | 5 | 5 | 5 | 5 | 5 |

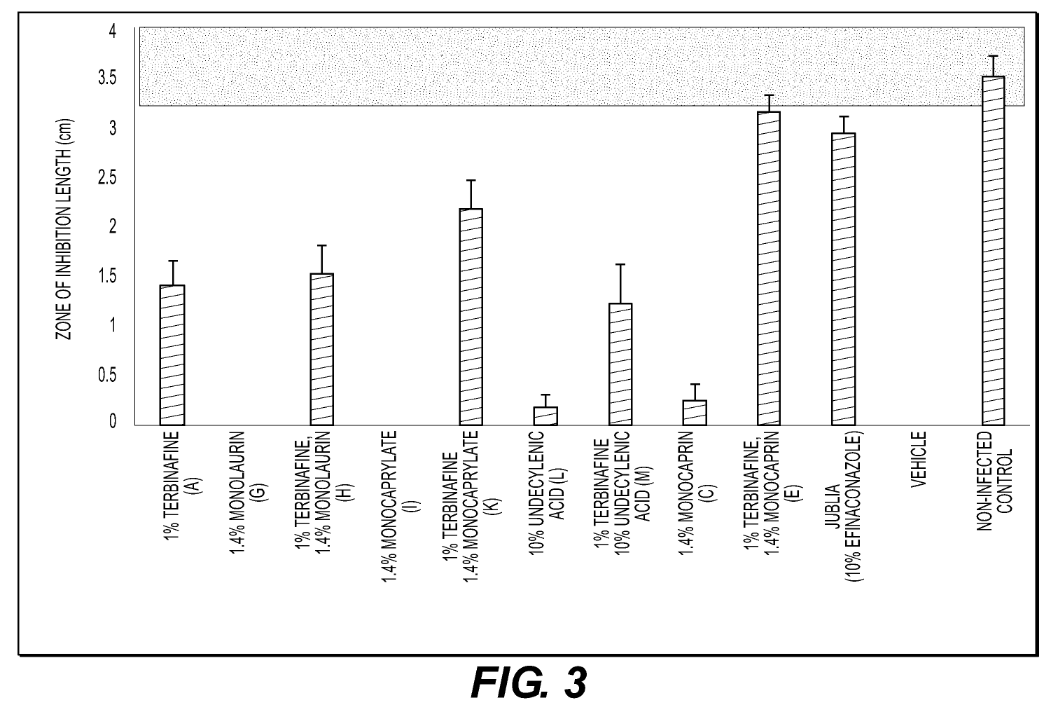

As shown in FIG. 3, the largest ZOI were observed for test formulations (E) (3.15 ±0.16 cm) and comparator Jublia® (2.93 ±0.17 cm). The rank of anti-fungal performance of the test formulations based upon the statistical significance of the mean ZOI distance was as follows: (E), Jublia® > (K) > (A), (H) > (M ) > ( C), (G), (L),( I), vehicle

When compared pair-wise, the ZOI for test formulation (E) (1% terbinafine, 1.4% monocaprin) was statistically greater than the combined zones of (A) (terbinafine 1%) and (C) (monocaprin 1.4%) indicating a synergic anti-fungal effect of the combination of terbinafine and monocaprin. However, the combined zone of inhibition test formulations (A) and (L) was greater than that of test formulation (M), indicating that combining undecylenic acid and terbinafine reduces anti-fungal efficacy rather than exerting the synergy observed for the combination of terbinafine and monocaprin.

The data indicate that monocaprin (C), monolaurin (G), and monocaprylate (I) alone at 1.4% concentration did not provide anti-fungal activity. Also, combining terbinafine with monolaurin or monocaprylate did not provide synergistic anti-fungal effects.

In summary, the trend in the data shown in FIG. 3 indicates that the optimal monoglyceride chain length is C₁₀ (monocaprin), as this confers a greater anti-fungal efficacy than monocaprylate (C_{B}) and monolaurin (C₁₂) in both the combined and single test formulations.

### Example 4

The Torchub® ZOI assay was used to evaluate the effect of varying the weight ratio of terbinafine to monocaprin in the test formulations shown on Table 4, using the solution chassis (“vehicle”) as a control.

Table 4

| Ingredients INCI (%w/w) | Test Formulation | | | | | |
|---|---|---|---|---|---|---|
| | (N) | (O) | (P) | (Q) | (R) | (S) |
| Terbinafine HCI | 2.81 [2.5] | 0.28 [0.25] | 0.28 [0.25] | 2.81 [2.5] | 1.55 [1.375] | 5.625 [5] |
| Monocaprin | 0.25 | 0.25 | 2.5 | 2.5 | 1.38 | 1.4 |
| solution chassis: | | | | | | |
| Ethyl alcohol, 100% v/v | 79.39 | 81.92 | 79.67 | 77.14 | 79.52 | 75.425 |
| Butylhydroxytoluene (BHT) | 0.05 | 0.05 | 0.05 | 0.05 | 0.05 | 0.05 |
| Stearyl alcohol | 0.5 | 0,5 | 0.5 | 0,5 | 0,5 | 0,5 |
| Cetyl alcohol | 1.1 | 1.1 | 1.1 | 1.1 | 1.1 | 1.1 |
| Polysorbate 60 | 0.4 | 0.4 | 0.4 | 0.4 | 0.4 | 0.4 |
| Propylene glycol | 10 | 10 | 10 | 10 | 10 | 10 |
| Sodium Hydroxide 30% (w/w) | 0.5 | 0.5 | 0.5 | 0.5 | 0.5 | 0.5 |
| Water | 5 | 5 | 5 | 5 | 5 | 5 |

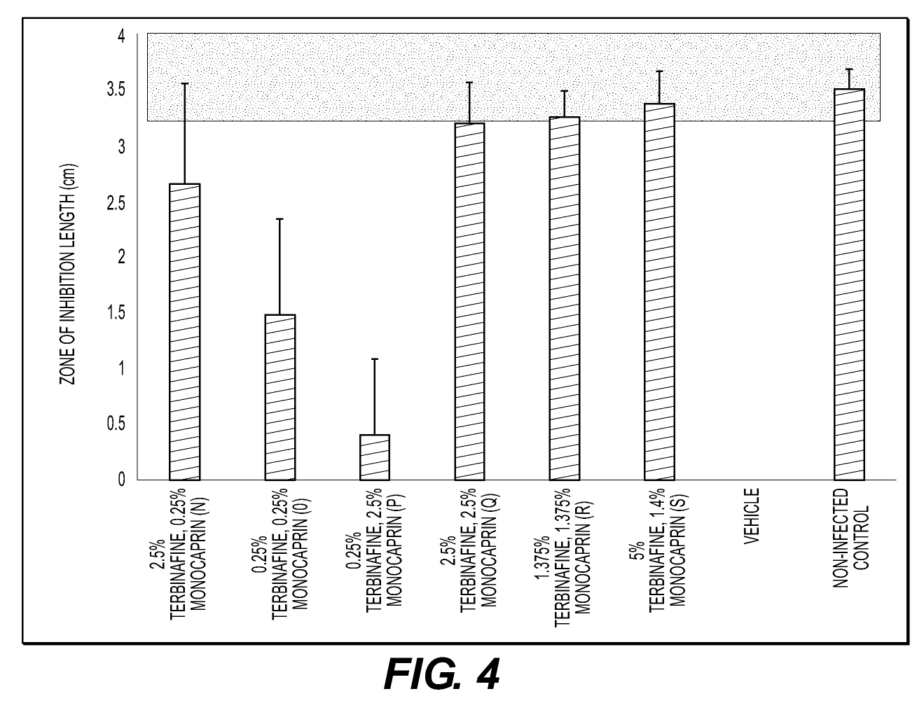

FIG. 4 shows the results of the ZOI study. The anti-fungal performance of the test formulations was ranked as follows, based on largest to smallest mean ZOI distance: (Q), (R), (S) (Full kill) > (N) > (O) > (P) > vehicle

At low terbinafine concentration (0.25%) decreasing the weight ratio of terbinafine to monocaprin below 1 resulted in reduced formulation anti-fungal activity.

The data indicate that in the above formulations superior anti-fungal efficacy was achieved when terbinafine concentration was about 1.38% or greater, with a terbinafine to monocaprin wt.% ratio of about 1 to 3.5, preferably about 1:1.

Test formulation (S) containing high levels of terbinafine (5% w/w) and medium levels of monocaprin (1.4% w/w) showed full kill of T. rubrum in the test system. However, there was no statistically significant difference among test formulations (Q), (R), and (S), indicating that by using the optimum weight ratio of terbinafine to monocaprin, similar anti-fungal activity can be achieved at lower levels of terbinafine and hence minimizing patient exposure to the anti-fungal drug and its potential side effects.

### Example 5

The ChubTur® infected nail model was used to investigate the anti-fungal efficacy of test formulations (A) and (E) of Table 2, against comparator Jubila® and vehicle. A single 2 µl dose of each formulation was applied to a nail and the nail was maintained at 2.5° C. for 24 hours.

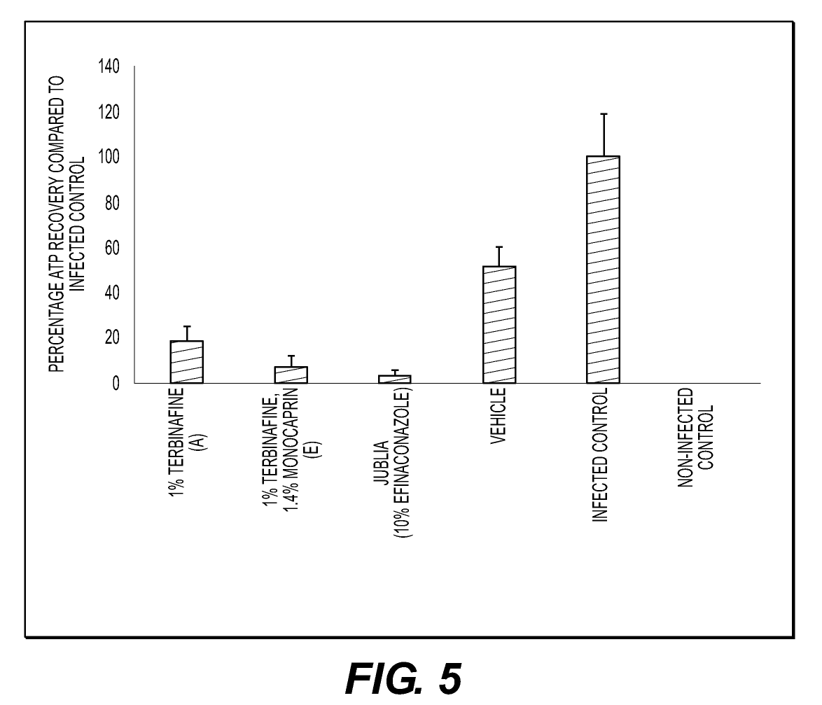

The mean ATP recoveries of each test formulation (compared to infected control) were as follows: 18.6% for formulation (A) [terbinafine (1% w/w) solution]; 7.0% for formulation (E) [terbinafine (1% w/w) and monocaprin (1.4% w/w) solution)], 3.4% for Jublia®), and 51.7% for vehicle, and are depicted on FIG. 5.

Test formulation (E) of the invention was statistically as efficacious against T. rubrum as the comparator, Jublia® (p > 0.05); both of which were also statistically similar to the non-infected control (p > 0.05), indicating complete kill of the organism.

While formulation (A) comprising terbinafine, 1% alone was efficacious against T. rubrum, this formulation was statistically different from the non-infected control (p 0.05), indicating that complete kill was not achieved. Some degree of anti-fungal efficacy was observed following application of the vehicle alone in this model.

Based on a statistical comparison of the ATP recovery in each case, the solutions were ranked as follows: (E) and Jublia® > (A) > Vehicle

In summary the data suggest that application of single dose of a composition of the present invention in an infected nail model can result in complete kill of T. rubrum.

### Example 6

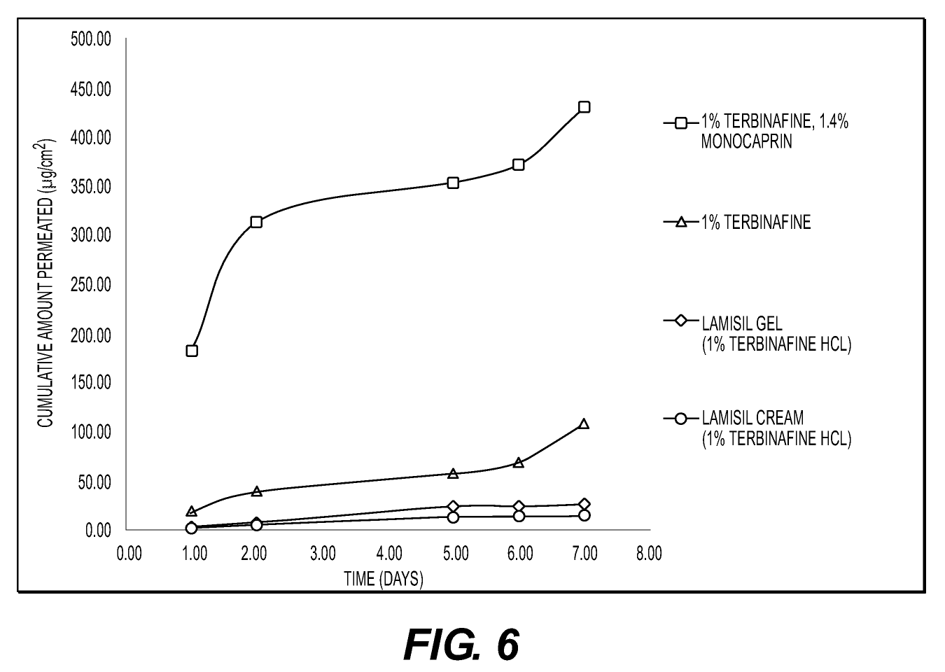

The permeation of terbinafine through bovine hoof, 70-90 µm in thickness, from comparators Lamisil® Dermgel, Lamisil® Cream, terbinafine 1% solution and a composition of the invention comprising terbinafine 1% + monocaprin 1.4% is shown in FIG. 6 (n=6 replicates for the active formulations and infected control, n=3 replicates for the placebo and non-infected control; mean ± SEM).

The cumulative amount of terbinafine permeated after 7 days is 127.5 µg/cm2. This is almost 9x greater than Lamisil® Cream and 4.9x greater than Lamisil® Dermgel.

The data correlate with ZOI results and indicate that the compositions of the invention efficiently deliver terbinafine across the nail.

### Example 7

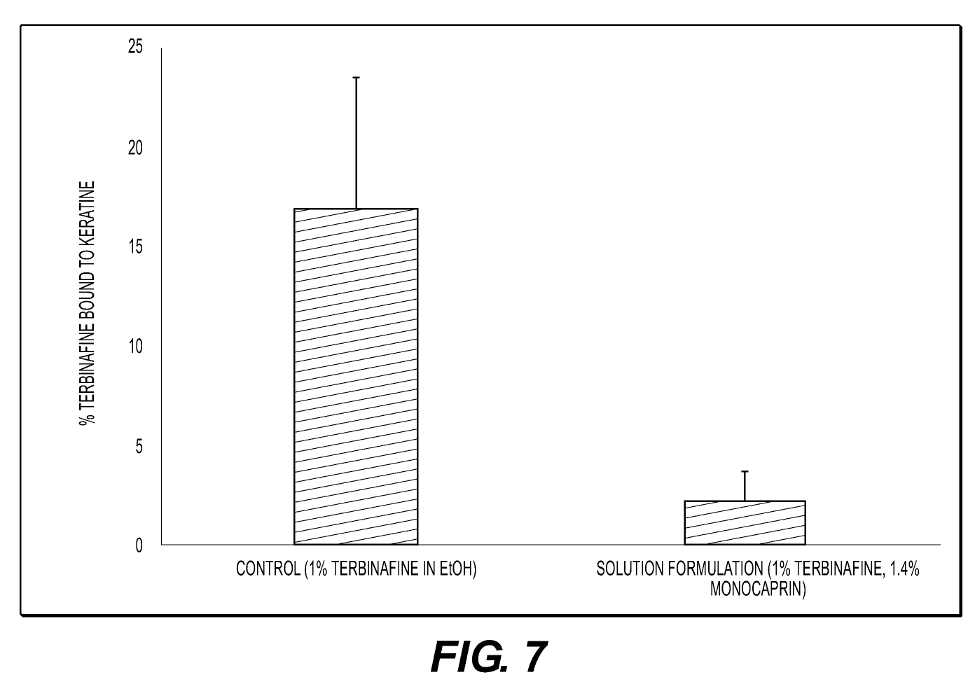

Drug keratin affinity is an important physicochemical property affecting the efficacy of anti-fungal drugs in onychomycosis, many of which such as terbinafine are reported to have high affinity to keratin hindering their ability to penetrate through the nail. FIG. 7 shows the percentage of keratin-bound terbinafine in 100% ethanol (control), and the present invention.

The data indicate that inclusion of monocaprin in the compositions of the invention significantly reduces the ability of keratin to bind to terbinafine. The free terbinafine can therefore penetrate through the nail and effectively reach the target site killing the fungus which is growing on the nail bed.

What is claimed is:

1. A topical pharmaceutical composition for the treatment or prophylaxis of onychomycosis, or other fungal conditions of the nail unit caused by Trichophyton rubrum and/or Trichophyton mentagrophytes, in a patient in need thereof, said composition comprising a therapeutically effective amount of an amine-based anti-fungal agent or pharmaceutically acceptable salt thereof, and monocaprin, in a wt.% ratio of 5:1 to 1:6 (amine free base: monocaprin), in a topically administrable vehicle.

2. A composition according to claim 1 wherein the amine-based anti-fungal agent is selected from allylamines and the pharmaceutically acceptable salts thereof.

3. A composition according to claim 2 wherein the amine-based anti-fungal agent is selected from terbinafine and the pharmaceutically acceptable salts thereof.

4. A composition according to claim 3 wherein the terbinafine and monocaprin are present in a wt.% ratio of 3.5:1 to 1:1.4 (amine free base: monocaprin) (based on the total weight of the composition).

5. A composition according to claim 3 wherein the terbinafine and monocaprin are present in a wt.% ratio of 1:1.4 to 1:5.7 (amine free base: monocaprin).

6. A composition according to claim 1 comprising 1 wt.% terbinafine (free base).

7. A composition according to claim 1 comprising 1 wt.% terbinafine (free base) and between 1.4 and 5.7 wt.% monocaprin.

8. A composition according to claim 1 wherein the topically administrable vehicle comprises at least one volatile solvent selected from the group consisting of ethanol, propyl alcohol, isopropyl alcohol, n-butyl alcohol, t-butyl alcohol, butoxy ethanol, acetone, ethyl acetate and butyl acetate, and mixtures thereof.

9. A composition according to claim 8 wherein the at least one volatile solvent comprises ethanol.

10. A composition according to claim 8 which further comprises one or more excipients selected from the group consisting of pH adjusting agents, humectants, film extenders, chelating agents, antioxidants, preservatives, plasticizers, penetration enhancers, fragrance, colorants, surfactants, emollients, gelling agents, and radical scavengers.

11. A composition according to claim 10 which comprises an emollient selected from C₁₂-C₁₅ alcohol lactates and cetyl alcohol.

12. A composition according to claim 1 which comprises a second pharmaceutically active agent.

13. A composition according to claim 12 wherein the second pharmaceutically active agent is selected from the group consisting of an antibacterial agent, anti-fungal agent, corticosteroid and vitamin D analogue.

14. A composition according to claim 1, wherein the fungal condition is selected from the group consisting of tinea pedis, tinea cruis, dermatophysis and onychomycosis.

15. A composition according to claim 1 comprising:
Terbinafine, 1 wt%
Monocaprin 1.4 wt.% or 5.7 wt.%
ethyl alcohol
butylhydroxytoluene (BHT)
stearyl alcohol
cetyl alcohol
polysorbate 60
propylene glycol
.

16. A method for the topical treatment or prophylaxis of onychomycosis, or other fungal conditions of the nail unit caused by Trichophyton rubrum and/or Trichophyton mentagrophytes, in a patient in need thereof, comprising topically applying to the affected area a composition according to claim 1.

17. A method according to claim 16 wherein the fungal condition is selected from the group consisting of tinea pedis, tinea cruis, and dermatophysis.

18. A method according to claim 16, wherein the fungal condition is onychomycosis.
